(19) United States
(12) Patent Application Publication
(10) Pub. No.: US 2024/0150705 A1
(43) Pub. Date: May 9, 2024

(54) POLYPHENOL INFUSED PROBIOTICS AND METHODS FOR IMPROVED GUT SURVIVABILITY, PERSISTENCE AND COLONIZATION

(71) Applicant: THE REGENTS OF THE UNIVERSITY OF CALIFORNIA, Oakland, CA (US)

(72) Inventors: Nitin Nitin, Davis, CA (US); Rewa Rai, Davis, CA (US); Marluci Palazzolli da Silva Padilha, Campinas (BR)

(73) Assignee: THE REGENTS OF THE UNIVERSITY OF CALIFORNIA, Oakland, CA (US)

(21) Appl. No.: 18/542,860

(22) Filed: Dec. 18, 2023

Related U.S. Application Data
(63) Continuation of application No. PCT/US2022/037513, filed on Jul. 18, 2022.
(60) Provisional application No. 63/223,126, filed on Jul. 19, 2021.

(51) Int. Cl. C12N 1/04; A61K 35/745; A61K 35/747; A61K 36/61; A61K 36/77; A61K 36/87; C12N 1/20; C12R 1/23; C12R 1/245; C12R 1/25

(52) U.S. Cl. CPC C12N 1/04; A61K 35/745; A61K 35/747; A61K 36/61; A61K 36/77; A61K 36/87; C12N 1/205; A61K 2236/333; C12R 2001/23; C12R 2001/245; C12R 2001/25

(57) ABSTRACT

Vacuum infusion of plant extracts in live probiotic cells significantly enhances the viability of probiotics during in vitro gastrointestinal digestion with the retention of their metabolic and antagonistic activities. In-vivo results validate the significantly enhanced persistence and mucoadhesion of probiotic cells with infused plant extracts.

### CROSS-REFERENCE TO RELATED APPLICATIONS

This application claims priority to, and is a 35 U.S.C. § 111(a) continuation of, PCT international application number PCT/US2022/037513 filed on Jul. 18, 2022, incorporated herein by reference in its entirety, which claims priority to, and the benefit of, U.S. provisional patent application Ser. No. 63/223,126 filed on Jul. 19, 2021, incorporated herein by reference in its entirety. Priority is claimed to each of the foregoing applications.

The above-referenced PCT international application was published as PCT International Publication No. WO 2023/003831 A1 on Jan. 26, 2023, which publication is incorporated herein by reference in its entirety.

### STATEMENT REGARDING FEDERALLY SPONSORED RESEARCH OR DEVELOPMENT

This invention was made with government support under Grant Number 2017-07600, awarded by the U.S. Department of Agriculture. The government has certain rights in the invention.

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

1. Technical Field

This technology pertains generally to beneficial microorganism supplementation and systems for delivery of sensitive material to the gut and more particularly to systems and methods for the production of fortified probiotics with improved survivability, persistence and capacity for colonization. It has been shown that probiotic cells infused with fruit and other plant extracts rich in polyphenols or purified polyphenols have significantly enhanced stability in the gastrointestinal tract. Simultaneous delivery of phenolic compounds (PC) and probiotics improve the proliferation of beneficial probiotics as well as the absorption of PC and their metabolites in the gut.

### 2. Background

Probiotics and plant-based diets have emerged as the leading food-based approaches to improving gut health. Probiotics are defined as living microorganisms that provide health benefits to humans when taken in adequate amounts, including improvements in gut and immune systems by balancing gut microbiota. In the case of probiotics, this improvement in gut health is associated with the successful delivery of probiotics to the colon and their ability to persist and generate beneficial metabolites such as short chain fatty acids, that can significantly influence both the gut and systemic health of the host.

With plant-based diets, key health benefits are associated with both the fiber content and phenolic compounds that are present in plants. A significant fraction of these phenolic compounds can be metabolized by gut microbes and the bioactivity of the resulting metabolites in the gut and in systemic circulation can significantly influence the gut microbiome that benefits the host.

One of the challenges of the application of probiotics in the form of food or supplements is the sensitivity of probiotics to the harsh conditions that can be used during the processing, storage and digestion of the supplement. Accordingly, delivery of probiotic bacteria and phenolic bioactives to the lower intestinal compartment of the gut and their ability to persist and interact with the gut microflora is a key constraint that limits their potential. The delivery of probiotic bacteria can be significantly limited by gastric and upper intestinal barriers including acidic pH in the gastric system, bile salts in the small intestine and a diversity of ambient proteases.

Because of these barriers, many researchers have attempted to select resistant strains or to protect the probiotics with microencapsulation. Among the encapsulation techniques, spray-drying has been widely explored for protection of probiotics. However, the technique has the deficiency of exposure of the probiotics to high temperatures during atomization. This process may influence probiotic viability due to the mechanical, thermal and oxidative stress created by the process. Thus, less invasive and stressful processes should be developed to improve the survivability of probiotics.

Pharmaceutical grade encapsulation systems rely on specific polymers for the gastric and intestinal protection of bacteria. Typical non-food grade polymers used for the gastric and intestinal protection include: polyethylene glycol, polyvinyl alcohol and cellulose acetate phthalate. Carbohydrate-based biopolymers such as alginates, chitosan, modified starches have also been developed for the encapsulation and delivery of probiotics. However, these polymers and their combinations were initially developed for the oral drug delivery of organic molecules and are not customized to address the unique attributes of living bacteria including factors that may promote the persistence and growth of these bacteria in the colon.

While these approaches have improved the viability of encapsulated bacteria in the gastric intestinal compartments, the release of probiotic bacteria from these capsules is complex. In addition, many of the conventional polymeric delivery systems provide limited benefits in promoting the persistence and growth of bacteria, that are essential for the biological function of these bacteria.

In addition, various combinations of prebiotic and probiotics have been evaluated by simply mixing freeze-dried bacteria with inulin and other prebiotics. In some cases, these formulations provided limited protection in the gastric and intestinal compartments. However, maintaining the viability of bacteria in these formulations prior to their oral delivery is also a significant challenge.

Some studies have evaluated the synergism between probiotics and phenolic compounds that may improve the proliferation of probiotics as well as the absorption of phenolic compounds in the gut. In particular, phenolic compounds were classified as plant-based prebiotics due to the observation that these compounds are bio-transformed by colon microbiota. In addition, polyphenols have important biological properties, such as the antioxidant, anticarcinogenic and antimicrobial activities.

Like probiotic supplementation, however, the delivery of phenolic bioactives to the lower intestine can be limited by chemical constraints. These constraints include exposure to a wide range of pH conditions, the limited solubility and hydrolytic breakdown of compounds, and the presence of biological barriers such as limited uptake of polyphenolics by target microbes.

In addition, the metabolism of phenolics by gut microbes requires key enzymes that have only been characterized in few classes of bacteria including Lactobacillus, Bacteroides and bifidobacteria. Thus, bioactivity of phenolic compounds significantly depends on the composition of the gut microbiome. Together, these chemical and biological barriers can influence the ability of gut microflora to metabolize plant phenolics and the generation of bioactive metabolites.

With increasing rates of the occurrence of metabolic diseases in the United States, there is a significant need to develop effective food-based solutions to address this societal challenge. Thus, there is an unmet need to improve delivery of both probiotic bacteria and plant phenolics using food grade approaches and improve conditions for the metabolism of plant phenolics by probiotic bacteria and gut microflora in the colon and increase their role in modulation of metabolic diseases.

### BRIEF SUMMARY

Fortified probiotics with improved survivability, persistence and capacity for colonization are provided for supplements as well as methods of production and use. The preferred constructions and methods address the challenges of enhancing delivery of both probiotics and plant phenolics to the gut and generation of beneficial plant phenolic metabolites.

In one preferred embodiment, probiotic cells are infused with fruit and plant extracts that are rich in polyphenols or purified polyphenols that also produces significantly enhanced stability in the gastrointestinal tract over cells that are not infused. This construction significantly enhances resistance of probiotic bacteria against acidic pH, proteases and bile salts thereby improving persistence of probiotic bacteria in-vivo.

The infused probiotics may not only improve the proliferation of beneficial probiotics but may also increase the availability and absorption of phenolic compounds and their metabolites in the gut. It is also important to note that after infusion the cells are stable and are metabolically active, thus the addition of polyphenols does not influence cell activity.

One feature of this approach is that it eliminates the need and deficiencies of conventional encapsulation processes and materials to protect probiotic and therapeutic microbes in the gut. The application of typically large biopolymers such as alginates and others to protect cells from acidic environment using encapsulation of cells in biopolymer matrix is unnecessary with infused cells. The methods transform the paradigm of using polymers and large macromolecules to protect probiotic bacteria from harsh conditions in gastro-intestinal compartments and to a plant phenolics infused based approach to protect and improve delivery of probiotic across gastro-intestinal barriers and to promote persistence of these bacteria.

Another feature of the construct and methods is that the infusion increases the hydrophobicity of the cell surface of the infused bacteria and thus improves their adhesion to gut walls and other microbial structures. This enhanced muco-adhesion helps with retention of the microbes in the gut.

A further feature of the constructs and methods is that it combines the co-delivery of plant-based prebiotics with protected probiotics. The benefits of delivery of phenolic bioactives to the lower intestine are often dependent on the composition of the gut microbiome and the availability of key enzymes that metabolize plant phenolics and generate useful bioactive metabolites.

An evolutionary bi-directional relationship exists between gut microbes and plant phenolic bioactives and that relationship benefits human health significantly. A large fraction (greater than 75%) of poorly absorbed plant phenolic compounds reaches the lower intestinal compartment where these phenolic bioactives are bio-transformed by resident microbiota. This bio-transformation is essential for absorption of these phenolic compounds in the gut and their bioavailability in blood circulation. Certain classes of gut bacteria such as Lactobacillus and Bifidobacteria spp. have developed specific set of enzymes for the metabolism of phenolic bioactives such as the glycosyl-hydrolases enzymes to enable the release of aglycones from glycol-conjugated phenolic compounds. Thus, bioconversion of phenolic compounds by gut microbes converts the poorly absorbed phenolic bioactives into a wide array of low-molecular-weight aromatic acids such as phenylacetic, phenylpropionic, phenylvaleric, and benzoic acid derivatives. These microbial-derived metabolites are more easily absorbed through the intestine, but also remain in the gut, where they may play a role in the maintenance of intestinal health. The ability to metabolize these phenolic compounds provides an advantage to certain classes of beneficial bacteria compared to many pathogens that are inhibited by the presence of phenolic compounds.

Infusion of one or more plant extracts into the surface membranes of beneficial bacteria is preferably performed with a vacuum infusion process, which is a fast, non-thermal and effective process compared with passive diffusion. Vacuum infusion of polyphenols in probiotic cells does not require high temperatures or the use of organic solvents, that both negatively influence probiotic viability, decreasing the oxidative stress of the cell by the infusion of polyphenols or other components of plant extracts.

Compositions developed from infused bacteria may include conventional carrier materials and tablet coatings. The infusion of plant phenolics derived from fruit skin extracts or purified polyphenolics into live probiotic bacteria will also influence the probiotic viability of the bacteria components from stress from freeze-drying and the formulation of the supplement.

In one preferred embodiment, sustainable sources of phenolic bioactives are used for synergistic combination with probiotic bacteria such as grape skin and grape seed phenolic extracts. Both the skin and seeds are part of the grape pomace and are a significant fraction of the by-products generated by the grape industry. For example, grape skin and/or seed phenolic bioactives have an established influence on gut health. It has been demonstrated that ingested polyphenols from grape pomace are modified by the gut microbiota and increased the numbers of polyphenolic metabolites were observed in the blood, urine, ileal fluid and feces. Intake of polyphenols also modulated the gut microbiota and contribute to beneficial microbial ecology that can enhance human health benefits. Thus, validating a two-way relationship between the gut microbiota and polyphenolic compounds from grape pomace.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a functional block diagram of a method for fabricating a supplement with live probiotic bacteria infused with plant extracts or purified polyphenolics according to one embodiment of the technology.

FIG. 2 is a plot of survivability of L. paracasei (LP), Lactobacillus casei (LC) and L. rhamnosus (LR) probiotic cells without infused plant extracts during simulated gastrointestinal treatments with simulated gastric fluid (SGF) for 2 h, followed by the treatment with simulated intestinal fluid (SIF) for next 2 h.

FIG. 3 is a plot of survivability of (LP), (LC) and (LR) probiotic cells infused with catechin during simulated gastrointestinal treatments with simulated gastric fluid (SGF) followed by simulated intestinal fluid (SIF).

FIG. 4 is a plot of survivability of (LP), (LC) and (LR) probiotic cells infused with Jaboticaba Peel (JP) extract during simulated gastrointestinal treatments with simulated gastric fluid (SGF) followed by simulated intestinal fluid (SIF).

FIG. 5 is a plot of survivability of (LP), (LC) and (LR) probiotic cells infused with Guarana Seed Extract (GSE) extract during simulated gastrointestinal treatments with simulated gastric fluid (SGF) followed by simulated intestinal fluid (SIF).

FIG. 6 is a plot showing survivability of probiotic bacteria without infused phenolics, with infused curcumin and fisetin, and with catechin simply mixed within simulated gastric fluid during simulated gastro-intestinal digestion.

FIG. 7 is a graph depicting metabolic activities of L. paracasei after exposure to in vitro gastric fluid expressed as fluorescence intensity by time.

FIG. 8 is a graph depicting metabolic activities of L. paracasei after exposure to in vitro gastric fluid expressed as fluorescence intensity by time.

FIG. 9 is a graph depicting metabolic activities of L. casei infused jabuticaba peel extract after exposure to in vitro gastric fluid expressed as fluorescence intensity by time.

FIG. 10 is a graph depicting metabolic activities of L. paracasei after exposure to in vitro gastric fluid expressed as fluorescence intensity by time.

FIG. 11 is a graph depicting metabolic activities of L. casei infused with catechin after exposure to in vitro gastric fluid expressed as fluorescence intensity by time.

FIG. 12 is a graph depicting metabolic activities of L. paracasei infused with catechin after exposure to in vitro gastric fluid expressed as fluorescence intensity by time.

FIG. 13 is a graph depicting metabolic activities of L. rhamnosus after exposure to in vitro gastric fluid expressed as fluorescence intensity by time.

FIG. 14 is a graph depicting metabolic activities of L. casei after exposure to in vitro gastric fluid expressed as fluorescence intensity by time.

FIG. 15 is a graph depicting metabolic activities of L. casei infused with fisetin after exposure to in vitro gastric fluid expressed as fluorescence intensity by time.

FIG. 16 is a graph depicting metabolic activities of L. rhamnosus infused jabuticaba peel extract after exposure to in vitro gastric fluid expressed as fluorescence intensity by time.

FIG. 17 is a graph depicting metabolic activities of L. casei infused with curcumin after exposure to in vitro gastric fluid expressed as fluorescence intensity by time.

FIG. 18 is a graph depicting metabolic activities of L. casei infused grape juice extract after exposure to in vitro gastric fluid expressed as fluorescence intensity by time.

FIG. 19 is a graph depicting metabolic activities of L. rhamnosus infused with catechin after exposure to in vitro gastric fluid expressed as fluorescence intensity by time.

FIG. 20 is a graph depicting metabolic activities of L. casei infused with fisetin after exposure to in vitro gastric fluid expressed as fluorescence intensity by time.

FIG. 21 is a graph of cell count for catechin infused bacterial cells compared to the controls observed in the small intestine (SI) at time points 5, 24 and 48 hours.

FIG. 22 is a graph of cell count for catechin infused bacterial cells compared to the controls observed in the cecum at time points 5, 24 and 48 hours.

FIG. 23 is a graph of cell count for catechin infused bacterial cells compared to the controls observed in the colon at time points 5, 24 and 48 hours.

FIG. 24 is a graph of cell count for catechin infused and control bacterial cells from ground mouse ilium tissue demonstrating enhanced mucoadhesion of cells with infused catechin.

FIG. 25 is a graph of cell count for catechin infused and control bacterial cells from ground mouse cecum tissue further demonstrating enhanced mucoadhesion of cells with infused catechin surfaces.

### DETAILED DESCRIPTION

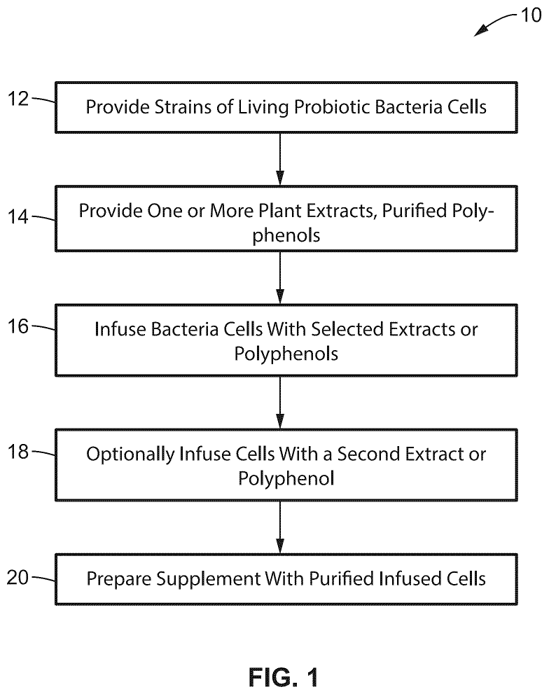

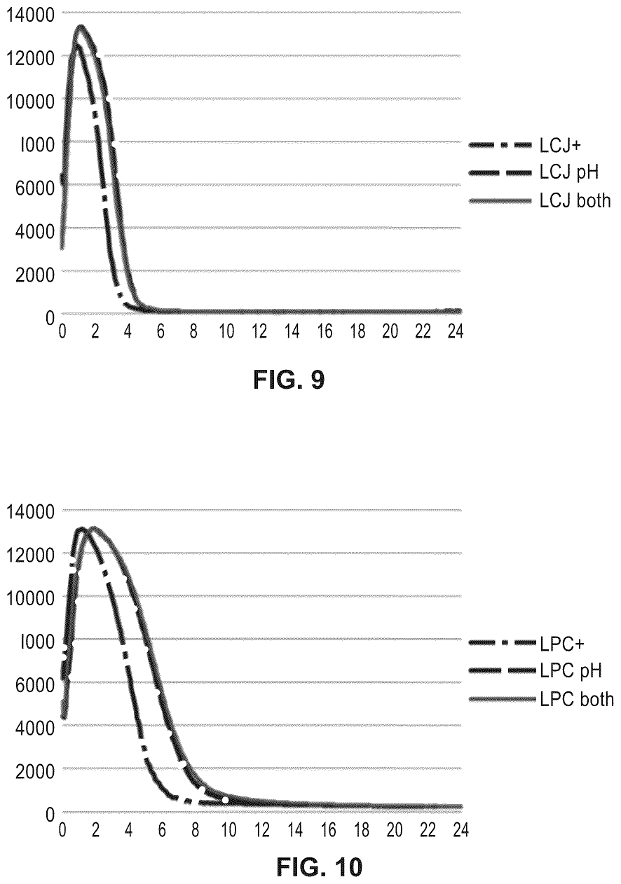

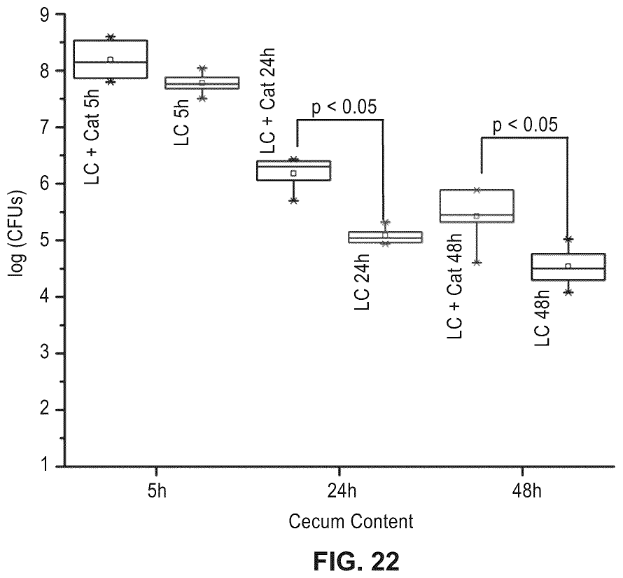

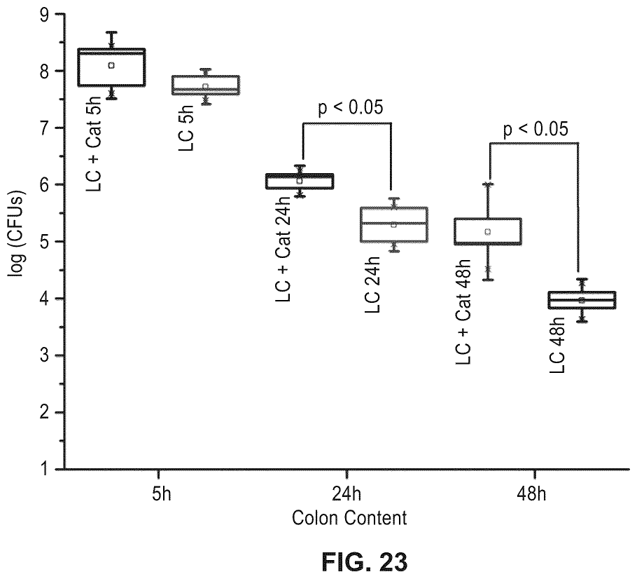

Referring more specifically to the drawings, for illustrative purposes, systems, devices and methods for producing beneficial probiotic constructs that have been infused with plant extracts that are rich in polyphenols, purified polyphenols or mixtures of extracts or different polyphenols and supplement compositions are generally shown. Several embodiments of the technology are described generally in FIG. 1 to FIG. 25 to illustrate the characteristics and functionality of the devices, systems and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology. The methods can be adapted to any situation where improved viability of different beneficial gut bacteria and supplementation with polyphenols and plant phenolic metabolites are desired.

Turning now to FIG. 1, an embodiment of the method 10 for assembling polyphenol or extract infused beneficial bacterial cells is shown schematically. At block 12, one or more beneficial bacterial types and strains are selected and acquired. There are many different beneficial probiotic strains that have been identified that may be selected at block 12 for fortification by the methods for enhanced stability, improved persistence and mucoadhesion of cells in the gastrointestinal tract. Bacterial strains of the genus Bifidobacterium, Lactobacillus, Bacteroides and Saccharomyces are particularly suited for selection for fortification with the methods. However, L. paracasei (LP), L. casei (LC) and L. rhamnosus (LR) are used to illustrate the methods.

Polyphenol rich plant extracts, purified polyphenols or mixtures of polyphenols are selected and prepared at block 14. Although polyphenol materials are preferred, it will be understood that other types of molecules or extracts that produce a protective effect when infused to increase survivability or longevity may be selected as well at block 14.

The selection of the polyphenol or mixture at block 14 may include matching and optimizing a specific polyphenol, mixture or extract with a particular beneficial bacteria strain. For example, a bacterial strain may be selected that has shown an ability to load a particular polyphenol or extract component that is desired for delivery. The specific polyphenol, mixture or extract may also be selected based on the observed protective effect for a particular bacterial strain or propensity for producing adhesion.

Preferred plant extracts, purified polyphenols or polyphenol mixtures for selection at block 14 are food grade or known to lack toxicity. Polyphenols are beneficial compounds that are present in many plant-based foods. There have been about 8,000 polyphenols grouped into about ten different classes and many different subclasses that have been identified and may be suitable for selection at block 14.

Polyphenols come from a variety of sources such as phenolic acids such as lignans and stilbenes that are found in vegetables and whole grains; flavonoids like quercetin, anthocyanins and catechins found in fruits; polyphenolic amides from peppers and grains, and others such as curcumin, resveratrol from wine and ellagic acid from berries.

In one embodiment, the plant derived phenolics and bioactives are obtained from sustainable sources. A variety of spent byproducts such as plant skins and seeds are generated by various food processing operations as waste. For example, grape pomace is a residue of grape processing in wine and juice, and it is estimated that it represents about 20% of the total weight of the fruit of the grape, which presents a challenging waste disposal problem for the winery and grape juice industry. Based on the compositional analysis, grape pomace is a rich source of plant phenolic bioactives including anthocyanins, catechins, flavonol glycosides, and phenolic acids. The content of the stilbenoid resveratrol, although small, is relevant as the price of the extracts often depends on the content of this minor constituent due to its demand and biological activity. Furthermore, these phenolic bioactives can be easily extracted from the spent byproducts using food grade solvents such as water-ethanol based extractions.

Another sustainable source of plant derived phenolics and bioactives is from Jabuticaba. Jabuticaba is a dark purple berry found in Brazil, which is consumed mainly as juice, jam, and ice-cream. The peel is removed because it may cause an astringency sensation. For this reason, jabuticaba peel is a waste by-product and a suitable source of polyphenols because most of bioactive compounds remain in the peel. Several studies investigating the potential of jabuticaba peel as a supplement for enrichment of foods have been conducted. Research has verified that jabuticaba peel extract (JPE) simultaneously applied with probiotics in a petit Suisse cheese formulation protected Lactobacillus acidophilus during processing and storage, but it did not affect the viability of Bifidobacterium lactis. Another study demonstrated that fruit by-product extracts can increase the growth of probiotic cells and their antioxidant activity may extend their health benefits. Thus, the production of a rich-phenolic extract from jabuticaba peel followed by the association with probiotic cells may be an alternative for simultaneous delivery.

Selected extracts or purified polyphenols are infused into the selected probiotic bacterial strain or strains at block 16 of FIG. 1. A negative pressure assisted infusion/encapsulation approach is preferred to infuse protective polyphenols into the bacteria. The overall objective of this approach is to enhance simultaneously the delivery of phenolic bioactives and probiotic bacteria using fortified bacteria supplementation.

The negative pressure assisted infusion/encapsulation is a non-thermal process that significantly enhances both the rate and encapsulated yield of bioactives in microbial cells. Infusion of phenolic bioactives into probiotics results in a multifold enhancement in resistance of probiotics to gastro-intestinal barriers. For example, the infusion of small molecules (mixture of polyphenolics or purified polyphenolic compounds) in cells protect the probiotic cells against the acidic environments of the gastric tract and also help with stability against bile salt. It is important to note that cells after infusion at block 16 are stable and metabolically active since the addition of polyphenols does not influence cell activity. Probiotics modified with phenolic infusion maintain their inhibitory activity against pathogens as illustrated by zone inhibition assays.

Optionally, a second type of plant extract, purified polyphenol or polyphenol mixture can be infused in the previously infused probiotic bacterial cells at block 18 of FIG. 1. The second polyphenol or polyphenol mixture may be selected at block 18 to provide synergistic or complimentary polyphenols with the first selection or to improve the growth environment or to specifically deliver a polyphenol. Simultaneous delivery of multiple types of phenolic compounds and probiotics may further improve the proliferation of probiotics and the absorption of the compounds and their metabolites in the gut. In addition, the second polyphenol or polyphenol mixture may be selected at block 18 to provide an increase in hydrophobicity of cell surface to enhance adhesion of the infused cells to the to the gut wall.

The infused or multiply infused probiotic bacteria are then purified and prepared for incorporation into a tablet or capsule at block 20 in the embodiment shown in FIG. 1. Supplement tablets or capsule compositions may include conventional pharmaceutical carriers and tablet coatings to facilitate storage and ease of consumption. Supplement compositions at block 20 may also include components that may assist in the metabolism, intestinal absorption and the bioavailability of the infused polyphenols.

The technology described herein may be better understood with reference to the accompanying examples, which are intended for purposes of illustration only and should not be construed as in any sense limiting the scope of the technology described herein as defined in the claims appended hereto.

### EXAMPLE 1

In order to demonstrate the functionality of the compositions and methods, three common probiotics, Lactobacillus casei, L. paracasei and L. rhamnosus were infused with jaboticaba peel extract (JPE) and guarand seed extract (GSE) and vacuum infusion and passive diffusion mechanisms were compared.

Extraction was performed using the proportion 1:25 (jabuticaba peel:solvent) at 40° C. and 200 rpm. First, dried jabuticaba peel was grinded by blender obtaining particle size below 0.1 mm. Water, 25% (v/v) ethanol and 50% (v/v) ethanol solutions were applied as solvent for extraction and designated as JPW, JP25 and JP50, respectively. Aliquots were removed during extraction (t=2 h, 4 h and 6 h) to evaluate the total phenolic content (TPC).

Guaraná seed extract (GSE) was produced using 25% ethanol solution and mechanical stirring at 60° C. for 30 min. The proportion of milled dried guarand seed and solvent was 1:20 (w/w).

Approximately 1 g of cells were added in 5 ml of plant extracts or catechin solution at a concentration 4 mg/mL in 25% ethanol and homogenized. As a control, 1 g of cells were added in 5 ml of 25% ethanol solution. Infusion of these plant extracts or a catechin solution in probiotic cells was conducted by vacuum and passive mechanisms. Passive diffusion was performed by keeping the samples under agitation at 7° C. for 24 h. This temperature was selected to maintain the viability of probiotic cells. Vacuum infusion was carried out according to conventional techniques, under conditions at 99% of vacuum for 5 s.

After infusion/encapsulation, all samples were washed three times using 5% ethanol solution to remove the remaining phenolic content. Supernatants were separated for quantification of total phenolic content.

Infused Lactobacillus cells with JP25% and GSE by vacuum were evaluated by multiphoton imaging. First, the plant extracts were scanned in a Leica TCS SP8 MP multi-photon microscope to check the wavelength of excitation (λ=800 nm) to view the bioactive compounds. Images of control and infused cells were collected in this condition. Microscopy of probiotic cells confirmed the infusion of JP25% and GSE in probiotic cells. In addition, samples were evaluated regarding the zeta potential before and after vacuum infusion. All measurements were performed in triplicate.

Vacuum infusion using jabuticaba peel in 25% of ethanol solution (JP25%) provided the maximum loading of phenolic compounds (PC), around 9 mg GAE/g of cells in less than 5 minutes without significantly influencing cell viability. On the other hand, loading of PC for passive diffusion was 2 to 4-fold lower than that obtained via vacuum infusion. Chemical composition of plant extract may have influenced the infusion of PC in probiotic cells, but the encapsulation was confirmed by multiphotonic microscopy.

Loading of phenolics of jabuticaba peel extract infused by passive diffusion ranged from 1.5 to 4.5 mg GAE/g of cells and exhibited values of encapsulation efficiency around 20%, regardless of the solvent applied for extraction of phenolic compounds from jabuticaba peel. Similar behavior was observed for GSE and catechin encapsulated by the passive mechanism, loading of TPC was 2 mg GAE/g of cells and encapsulation efficiency was around 8%. Other adjustments during infusion, such as changes in pH, different concentrations of bioactive compounds, etc., could increase the loading of phenolics and encapsulation efficiency.

Samples were freeze-dried after encapsulation to evaluate the viability of probiotic cells during storage. In relation to the viability of probiotic cells, 0.1 g of infused cells or freeze-dried cells were added in 5 ml of PBS buffer followed by serial dilution. Aliquots were removed in each tube and placed onto MRS agar. Plates were incubated at 37° C. for 2 days.

Viability of probiotic cells after encapsulation and freeze-drying was evaluated. As expected, freeze-drying was a mild process, showing probiotic population count at least 8.2 log CFU/g. Viability of freeze-dried cells vacuum infused with plant extracts was between 5.8 to 10.5 log CFU stored at room temperature up to 30 days. Probiotic cells loaded with GSE and catechin exhibited higher values of viability during storage, ranging from 8 to 10 log CFU.

Stability of encapsulated JPE25% was performed after freeze-drying and during storage up to 30 days at 25° C. Here, 0.1 g of freeze-dried cells was added in 2 mL of acidified methanol solution (70% v/v) and approximately 10 mg of zirconia beads. Samples were vortexed and ultrasonicated for 5 min each step, followed by centrifugation at 10,000 rpm for 3 min.

Among the mechanisms, vacuum infusion enhanced the total polyphenol content in probiotic cells and probiotic viability in addition to being a fast process compared with passive diffusion. Furthermore, chemical composition of plant extracts may also play a role for encapsulation.

### EXAMPLE 2

To further demonstrate the methods and the variety of polyphenols and plant extracts from sustainable sources, phenolics were extracted from grape pomace using ethanol-water extraction and characterized using an analytical HPLC analysis with known standards of phenolic bioactives. Briefly, syrah grape seed and dried grape skin powders respectively, were extracted using a 75% ethanolic solution. Chromatography separation and detection of phenolic compounds were performed using an Agilent 1260 Infinity II HPLC system (Santa Clara, CA) equipped with a temperature-controlled auto sampler, column compartment, a diode array detector (DAD) and an Agilent PLRP-S 100 Å (4.6×150 mm, 3 μm) column with an Agilent 3×5 mm guard column at 35° C. The flow rate and the injection volume were 1 ml/min and 10 μl, respectively. A 1.5% phosphoric acid (solution A) and an acetonitrile (Solvent B) containing 20% (v/v) solvent A were used. The gradient protocol was: 0 min, 94% solvent A; 73 min, 69% A; 78 min, 38% A; and 90 min, 94% A. Absorbance spectra for all peaks was recorded from 250 nm to 600 nm. External calibration curves were prepared with authentic standards of known phenolic compounds and used for quantification of the major extracted compounds. Polymeric phenols from the pomace extract were quantified based on catechin equivalents.

The phenolics extracted from grape spent materials and purified major compounds available in these spent materials were individually infused in the probiotic cells using negative pressure assisted infusion of bioactives. Well characterized probiotic bacteria from Lactobacilli (L. plantarum ATCC 14917 and L. rhamnosus GG (LGG ATCC 53103) and Bifidobacteria (Bifidobacterium longum ATCC 15707 and Bifidobacterium bifidum ATCC 29521), families were selected because these bacterial strains are extensively used in food products. These probiotics are also selected because prior research has demonstrated bioconversion of phenolic bioactives compounds by both Lactobacillus and Bifidobacterium.

Polyphenols extracted from grape seed and skin part using 75% ethanol were diluted with water to a final concentration of 25% v/v ethanol concentration in the mixture. Stock solutions of purified compounds were prepared in ethanol and diluted to make a 25% of ethanolic solution prior to infusion.

The selected bacterial strains were added to the diluted plant extract and the purified polyphenolic solutions. The mixture was then subjected to a negative pressure (90% of absolute vacuum level) for 5 s to infuse polyphenols into the probiotic bacteria as illustrated in Example 1. After infusion, the cells were pelleted down and washed twice using 1×PBS and freeze dried for further use.

To maintain anaerobic condition during washing, 1×PBS was degassed prior to use and tube tightly closed after purging with inert gases to the headspace. To quantify the infused content of plant extracts in the microcarriers, phenolic content in the aqueous-ethanol solution before and after infusion process was quantified with HPLC.

To quantify the microbial count of viable cells after infusion of plant extracts and freeze-drying process, the standard plate counting methods were used. Viable Lactobacillus cells were enumerated by spread plating on the de Man Rogosa Sharpe medium (MRS) and plating Bifidobacterium strain on the BSM agar plates. Plates were incubated at 37° C. in GasPak Anaerobe Pouches for 48-72 h. The viability (Log CFUs/ml) was calculated and the localization of infused polyphenols in cells was visualized using multiphoton fluorescence microscopy.

Metabolic activity of cells with and without infused plant extracts and after simulated digestions was evaluated using the resazurin fluorescence assay. For this assay, 6 log CFUs/ml of cells (with and without infused phenolic actives) in selective culture media will be prepared. 1 mL of each samples will be added to a flat bottom 24 well culture plates and 50 μM of resazurin was added to each well plates. Plates were sealed with PCR sealing films inside anerobic cabinet to maintain in-situ anaerobic conditions and then immediately incubated at 37° C. for 16 h. Fluorescence data was collected each 5 min at 580 nm (λₑₓ=530 nm).

### EXAMPLE 3

The survivability of infused cells and their interactions with the gastrointestinal environment during simulated digestion tests was demonstrated. To illustrate the role of phenolic bioactives in influencing cell viability, changes in the intracellular pH and interactions of bile salts with cells were measured after simulated gastric and intestinal treatments respectively. Survivability of cells with and without infused plant extracts were evaluated during simulated gastrointestinal treatments under anaerobic conditions. Simulated gastric fluids (SGF, pH=3) were prepared by adding 0.5% w/v sodium chloride, hydrochloric acid to maintain pH and 3.2 mg/ml pepsin. 1 g of selected bacterial cells with and without infused plant extracts were separately incubated in SGF for 2 h at 37° C. and 100 rpm.

After gastric treatment, the cells were pelleted down by centrifuging at 5000 rpm for 2 min and then resuspended in the simulated intestinal fluids (SIF) containing 3.33 mg/mL calcium chloride, 5 g/L sodium chloride, 10 mM bile salts and pancreatin (100 U/ml) in 0.025 M potassium phosphate buffer (pH=7). Digestion mixtures were incubated at 37° C. and 100 rpm for 2 h. After the simulated gastric and intestinal digestions, both the phenolic infused cells and control cells were isolated by centrifuging at 5000 rpm for 2 min and will be washed three times with autoclaved 1×PBS. Viable cells were enumerated after serial dilutions on selective media plates. The viability of cells were measured in triplicates and the results of these measurements were averaged.

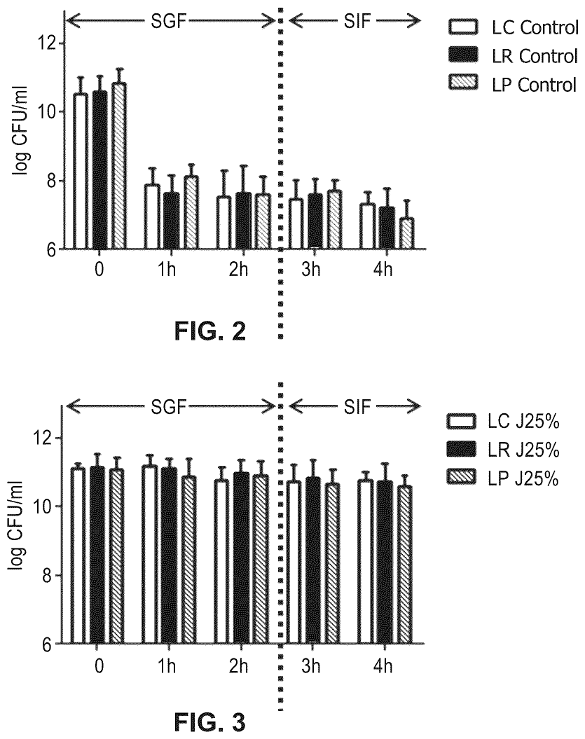

Live probiotic cells must reach the colon alive for colonization and related health benefits. The survivability of probiotic cells during in vitro digestion tests is shown in FIG. 2 through FIG. 5. These figures plot survivability of L. paracasei (LP), Lactobacillus casei (LC) and L. rhamnosus (LR) probiotic cells with and without infused plant extracts during simulated gastrointestinal treatments with simulated gastric fluid (SGF) for 2 h, followed by the treatment with simulated intestinal fluid (SIF) for next 2 h.

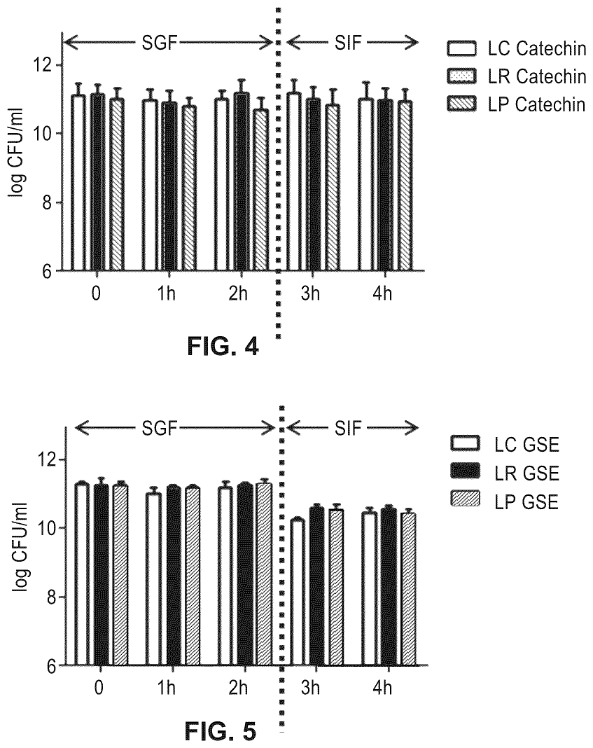

The LP, LC, and LR probiotic cells without infused plant extracts are shown as a control in FIG. 2. Cells infused with only ethanol 25% (control) reduced around 2 log CFU after the addition of SGF and completing to 3 log CFU by the end of the in vitro test. FIG. 3 is a plot of survivability of (LP), (LC) and (LR) probiotic cells infused with catechin, FIG. 4 plots survivability of probiotic cells infused with Jaboticaba Peel (JP) extract and FIG. 5 is a plot of survivability of probiotic cells infused with Guarana Seed Extract (GSE) extract.

Upon treating these probiotic cells with infused phenolic bioactives, significant enhancement in the survivability of probiotic bacteria during simulated gastro-intestinal digestion could be seen. A comparison of the results in FIG. 2 through FIG. 5 demonstrates approximately 10³ fold improvement in survivability of three different strains of Lactobacillus cells with infused phenolic bioactives (LC J25%, LR J25% and LP J25%) after the simulated gastric and intestinal digestions as shown in FIG. 4. These results suggest the key role of phenolic bioactives in improving the resistance of probiotic cells against harsh bio-chemical conditions during a simulated gastro-intestinal digestion.

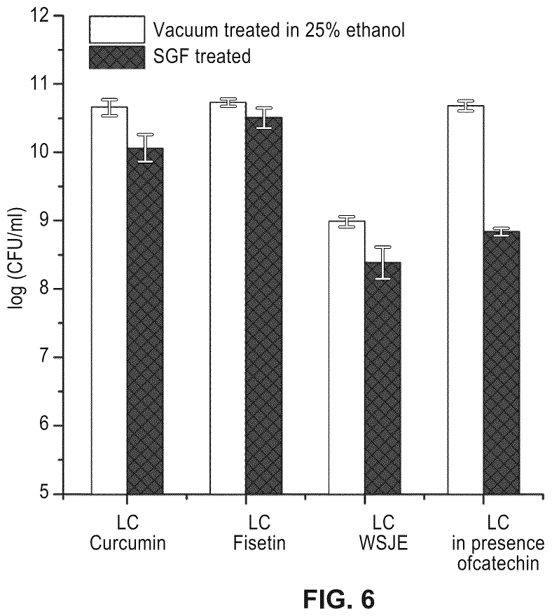

Like catechin, the jaboticaba and guarana seed extracts were stable after the addition of simulated gastric fluid, as well as simulated intestinal fluid. Enhanced stability of cells with infusion of plant extracts were further validated by evaluating the effect of gastric treatment on the survivability of model probiotic L. casei cells loaded with other hydrophobic polyphenols such as curcumin and fisetin and hydrophilic fruit extracts (e.g., WSJE in FIG. 6). Survivability of probiotic cells was not improved if polyphenols were not infused in cells but only added in the same amount to the gastric fluid.

In addition, jabuticaba peel exhibited prebiotic activity like the fructooligosaccharides (FOS), depending on the probiotic strain. For this co-encapsulation of probiotic and polyphenols in polymeric matrices could be protective. Plant extracts may reduce the oxidative stress or inhibited enzymatic activities avoiding the death of probiotics. As previously noted, GSE is an inhibitor of α-glucosidase and lipase.

To further validate that the observed phenomenon was not limited to this complex composition of phenolic extract from the skin of the tropical fruit (jabuticaba), preliminary studies using a purified phenolic compound (Catechin). Catechin was selected as it is reported to be one of the key components of the phenolic extract from this selected fruit. The results in FIG. 3 and FIG. 6 validates that indeed infusion of catechin in probiotic cells (LC Catechin, LR Catechin, and LP Catechin) can significantly enhance survivability of probiotic bacteria during a simulated gastro-intestinal digestion.

The potential of other phenolic compounds such as fisetin (flavonoids) and curcumin were also infused and tested. The results shown in FIG. 6 support the earlier observation that infusion of phenolic bioactives can significantly enhance survivability of probiotic bacteria during simulated gastro-intestinal digestion. This improvement in survivability by infusion of bioactives in probiotic cells was also validated by designing a control study in which purified catechin was simply mixed with probiotic cells and subjected to gastro-intestinal digestion. The results of this control study demonstrated no significant improvement in survivability of probiotic bacteria by a simple mixture of phenolic bioactives and probiotics during a simulated digestion. These results further validate the unique food grade composition developed by the infusion of phenolic bioactives in probiotic cells to enhance their resistance against gastro-intestinal conditions.

### EXAMPLE 4

Metabolic activity and pathogenic inhibitory function of phenolic infused probiotics was illustrated. In addition to survivability, the results of preliminary studies indicated that the infusion of phenolic bioactives (catechin infused L. casei) in probiotic bacteria did not significantly influence the metabolic activity of the bacteria nor their ability to suppress the growth of pathogenic organisms.

To further demonstrate that the infusion of phenolic bioactives does not significantly inhibit the probiotic activities of these bacteria, the metabolic activity of cells with and without infused plant extracts and after simulated digestions were evaluated using the resazurin fluorescence assay. For this assay, 6 log CFUs/ml of cells (with and without infused phenolic actives) in selective culture media was prepared. 1 mL of each of the samples were added to a flat bottom 24 well culture plates and 50 μM of resazurin was added to each well plates. Plates were sealed with PCR sealing films inside anerobic cabinet to maintain in-situ anaerobic conditions and were immediately incubated at 37° C. for 16 h. Fluorescence data was collected each 5 min at 580 nm (λₑₓ=530 nm).

The reduction of resazurin was evaluated by the assay. After cells were treated with simulated gastric fluid, 1 mL of each samples was centrifugated (16,000×g for 2 min) and resuspended in 1 mL of MRS broth containing 50 μM of resazurin. Then, plate was incubated at 37° C. for 16 h and read each 5 min (530 nm/580 nm). As a control, cells did not treat with simulated gastric fluid were evaluated.

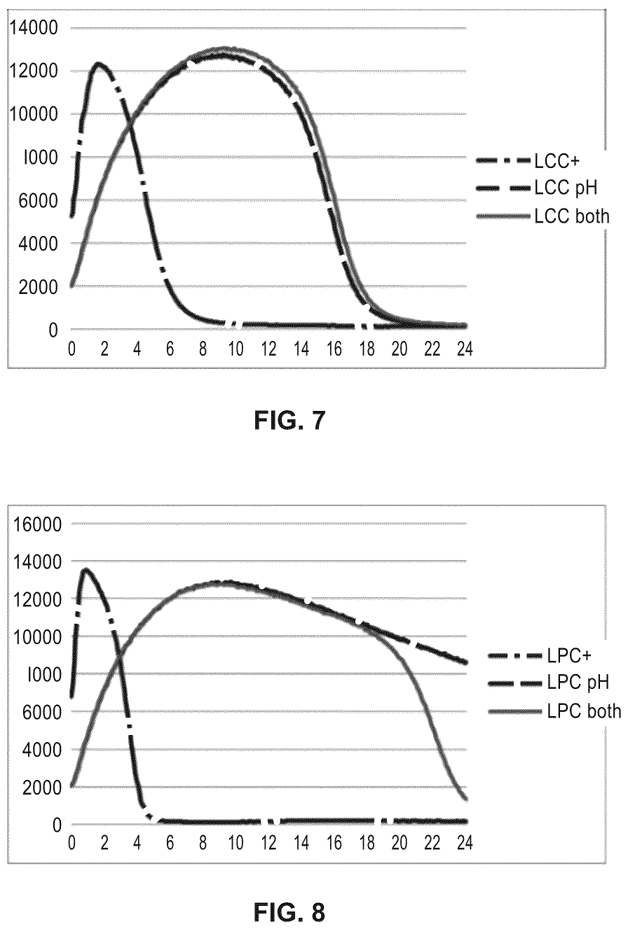

The reduction of resazurin to resorufin (fluorescent form) was measured to correlate with the metabolic activity of three types of probiotic cells with different infused polyphenols and conditions, as shown in FIG. 7 through FIG. 20. The production of resorufin increases the intensity of fluorescence, while the reduction of the peak is related with the production of hydroresorufin.

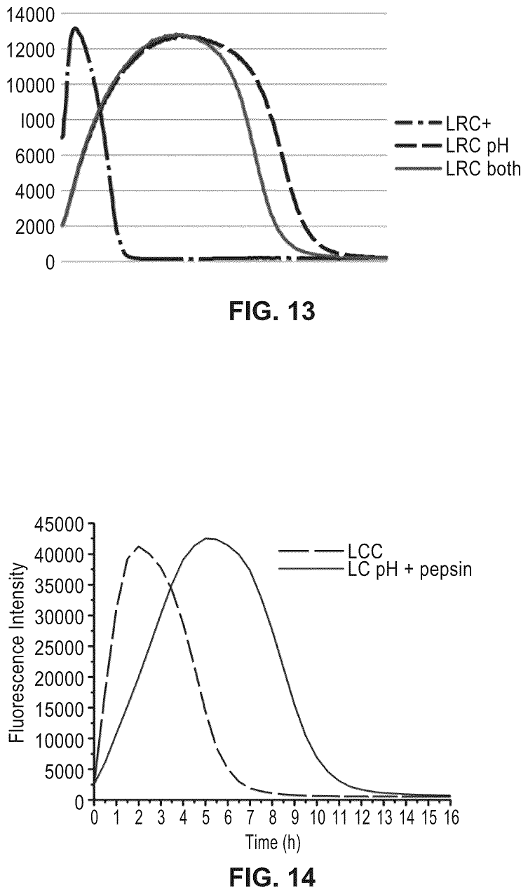

Metabolic activities of L. casei, L. paracasei and L. rhamnosus cells infused with plant extracts after in vitro gastric fluid, expressed as fluorescence intensity by time are shown in FIG. 7 through FIG. 20. Maximum fluorescence intensity for control cells (not treated with SGF) shown in FIG. 7, FIG. 8 and FIG. 13 for LC, LP and LR was achieved around 1-2 h of test. Cells treated with SGF for 2 h showed a delay to get the maximum intensity of approximately 8 h. However, infused cells with most of the plant extracts treated with SGF showed the maximum intensity at the same time required for cells not treated with SGF.

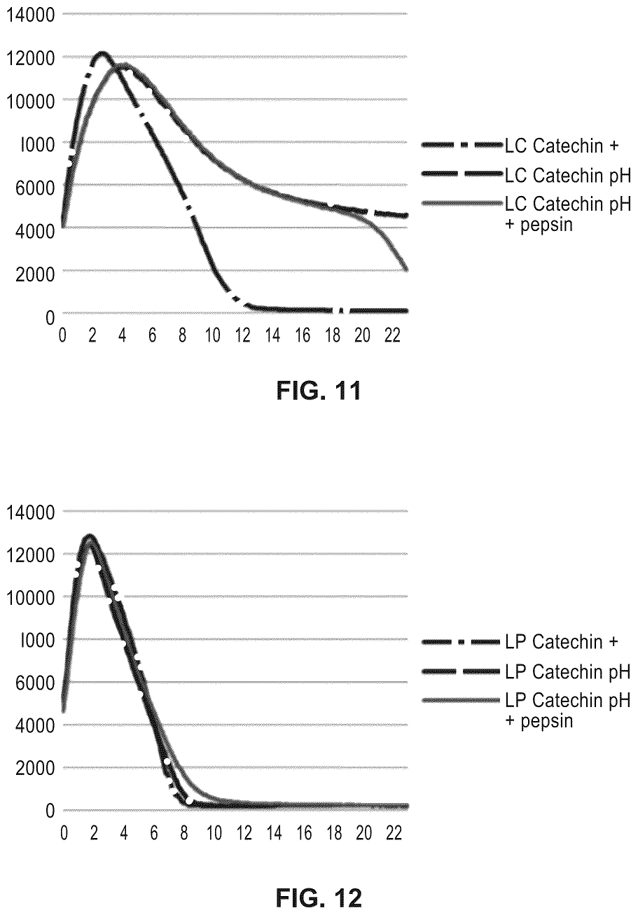

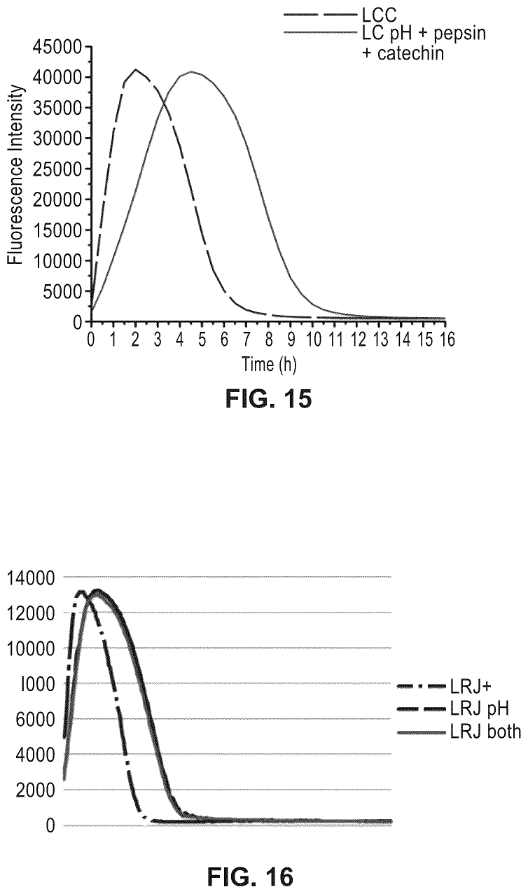

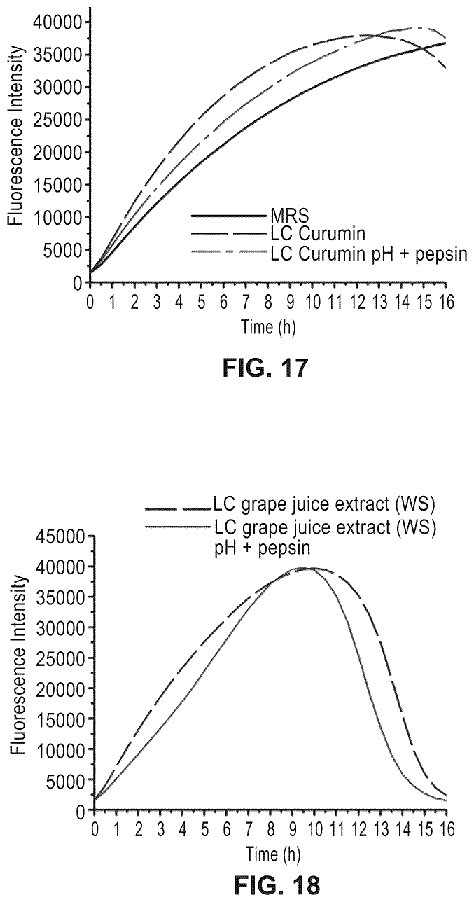

Metabolic activity of L. casei with and without infused catechin after simulated gastrointestinal digestions is shown in FIG. 7, FIG. 11 and FIG. 15. The metabolic activity of L. casei with infused with curcumin or fisetin after simulated gastrointestinal digestions is shown in FIG. 17 and FIG. 20 respectively.

The metabolic activity of L. paracasei with and without infused catechin after simulated gastrointestinal digestions is shown in FIG. 6, FIG. 10 and FIG. 12.

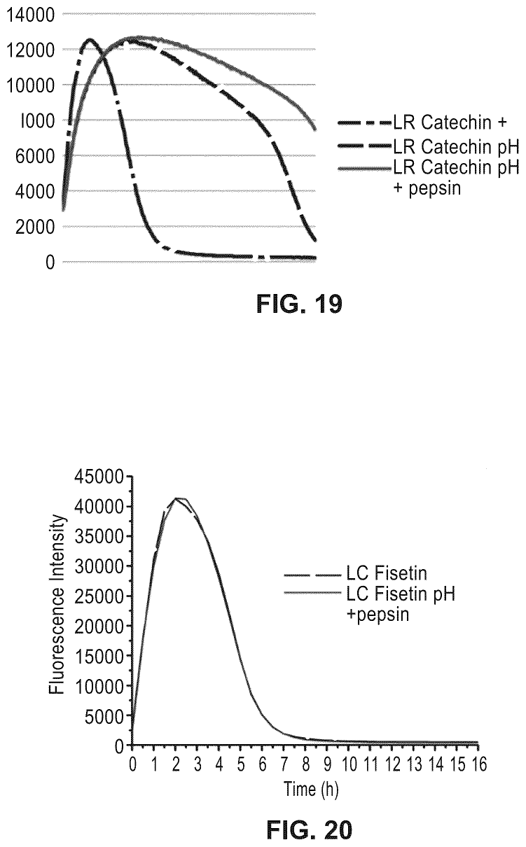

Metabolic activity of L. rhamnosus with and without infused catechin after simulated gastrointestinal digestions is shown in FIG. 13, FIG. 16 and FIG. 19.

Infusion of curcumin shown in FIG. 17 and water-soluble grape juice extract shown in FIG. 18 shows that cells change their metabolic activity as the maximum intensity was achieved at 8-10 h. However, the metabolic activity of cells with infused water-soluble grape juice extract was retained after simulated gastric treatment.

Metabolic activity of cells infused with curcumin was further suppressed with the simulated gastric treatment as seen in FIG. 17. The suppression of metabolic activity of cells infused with curcumin is obvious as curcumin is well known as an antibacterial and shows cells metabolic inhibition at certain concentration.

The maintenance of antagonistic activity against pathogens of infused probiotics was also analyzed. Antagonistic test of probiotics with and without infused plant extracts against indicator bacterial strains (Escherichia coli O157:H7, ATCC 700728 and Listeria innocua, ATCC 33090) was evaluated using agar spot method (dos Santos et al., 2019). L. casei strain and catechin were selected as the model probiotic cell and plant extract for this test. The Lactobacillus strain (viable count 10 log CFU/ml) with and without infused catechin were spotted (10 ul for each spot, 4 spots per plate) on MRS agar plates. A solution of Lactobacillus strain supplemented with catechin and catechin only were also spotted on MRS agar to evaluate the effect of catechin without infusion. Spotted plates were incubated anaerobically at 37° C. for 24 h. At the end of incubation, BHI agar solution (18 ml) mixed with 1 ml aliquot of indicator bacterial culture (viable count 9 log CFU/ml) were poured over the spot-inoculated MRS agar plates. Plates were incubated aerobically at 37° C. for 48 h.

Antagonistic activity of model probiotic cell L. casei infused with model plant extract catechin against pathogenic strains L. innocua and E. coli were evaluated using spot method shows similar antagonistic response of model probiotic cell L. casei with and without infused catechin. These results in combination with metabolic response and survivability validates that infusion of plant extracts in probiotic cells significantly enhance cells tolerance against simulated gastrointestinal treatments without any change in their metabolic or antagonistic activities.

### EXAMPLE 5

The muco-adhesion properties and cell surface hydrophobicity of probiotic cells infused with and without phenolic bioactives were evaluated to demonstrate the controlled improvements from infusion. Hydrophobicity and total protein content were evaluated by the Lowry method.

Probiotic cells were activated in 10 mL of MRS broth at 37° C. for 18 h. Then, cells were added in 100 mL of MRS broth. Finally, cells were collected by centrifugation and washed twice with sterile PBS buffer. Vacuum infusion of polyphenols was carried out using the ratio 1:5 (w/w) of probiotic cells to extract solution. Then, the mixtures were treated with 99% of vacuum for 5 s. Infused cells were washed three times with PBS buffer, removing the remained extracts.

Cells before and after the vacuum infusion were washed twice and resuspended in sterile PBS buffer. The bacterial suspension was adjusted by optical density (OD) at 640 nm to around 0.8. After, 3.5 mL of sample was mixed with 0.5 mL of octan using a vortex for 2 min. The tubes were rested, and phases were separated after 15 min. Aqueous phase was removed and the OD was determined at 640 nm. Cell surface hydrophobicity was calculated with:

Hydrophobicity(%)=[(0D₀−0D_{F})/0D₀]×100

Total protein content was evaluated following the Lowry method. The solution A was composed by sodium citrate at 2 g/L and sodium carbonate at 100 g/L in 0.5 M NaOH, while solution B contained 0.2 g/L of sodium citrate, 0.1 g/L of copper sulfate and 0.1 M of NaOH (after dilute copper). Solution C was prepared diluting 1 mL of Folin Ciocalteu reagent in 15 mL of distilled water.

Proteins from probiotic cells were extracted using NaOH 1 M and heating. Cells were added in 0.5 mL of 1 M NaOH in 3.5% NaCl and vortexed for 5 min. Approximately 0.1 g of silica bead was added and vortexed again for 5 min. Tubes were heated 60 C for 1 h and centrifugated. Then, 0.1 mL of extract was added in 0.9 mL of solution A and incubated at 50 C for 10 min. Next, 1 mL of solution B was added in the mixture and waited 10 min. Finally, 3 mL of solution C was added followed by the incubation at 50 C for 10 min. Absorbance was read at 650 nm.

Binding properties of cells are related to the hydrophobicity and total protein content. These parameters correlated with the profile of bioactive compounds, mainly log p, may indicate the potential of probiotic cells to bind vegetable extract. Table 1 shows the total protein content and hydrophobicity of probiotic cells before and after vacuum infusion.

As expected, total protein content of probiotic cells did not differ significantly. Hydrophobicity of probiotic cells ranged from 5.7 to 16%, even after the vacuum infusion of 25% ethanol solution. However, cells loaded with catechin or vegetable extracts increased the hydrophobicity, which is desirable to improve the bind in the cell wall gut.

Mucin binding properties can enhance retention of probiotics in the colon. The mucin-binding properties of probiotic cells infused with phenolic bioactives were evaluated and compared with the control probiotic cells. For this binding analysis, cells were stained with SYBR green dye after simulated gastrointestinal digestion. The stained cells were added to the mucin from porcine stomach (type II, 15 mg/ml) stained with Alexa 647 dye (5 μM). The suspensions were incubated for 20 min. After incubation, the mucin gel was imaged and based on the fluorescence intensity, the relative binding affinity of the probiotic cells with and without infused phenolic bioactives were characterized.

The cell surface hydrophobicity can significantly influence adhesion of bacterial cells to mucin. Bacterial surface hydrophobicity was also evaluated using the cell adherence to a hydrocarbon assay. Under anaerobic conditions, cells with infused phenolics were suspended in 1×PBS with final OD600 of 0.8. 1 ml of octane was be added to test tubes containing 3 mL of cells suspension. The mixture was blended on a vortex mixer for 90 s. The tubes will be left to stand for 15 min for phase separation. The OD₆₀₀ of the aqueous phase was measured. Hydrophobicity was calculated from three replicates as the percentage decrease in the optical density of the original bacterial suspension due to partitioning of cells into a hydrocarbon layer.

The in-vitro muco-adhesion properties based on cell surface properties of the infused probiotic cells were also characterized using gene expression analysis. Gene expression of probiotic cells for stress response and muco-adhesion genes was analyzed with selected strains of L. plantarum ATCC 14917 and Bifidobacterium bifidum ATCC 29521, as these strains have been sequenced. In this analysis, the transcriptional level of probiotic cell strains genes associated to: (i) stress response, i.e. encoding molecular chaperones (dnaK, groEL), and stress factors (ftsH) in selection probiotics; and to (ii) colonization and probiosis, i.e. mucin-binding protein (mub) and mannose adhesin (msa) in Lactobacilli and transaldolase (tal) in bifidobacteria were evaluated using real-time quantitative PCR. The sequences of specific genes were retrieved from PubMed/ATCC and primers were designed using Primer3Plus software.

Total RNA was extracted from cells before and after simulated gastro-intestinal digestion using the TRIzol plus RNA purification kit and cDNA was synthesized using a Gene Amp RNA PCR kit. For this analysis, cells infused with plant extracts and purified compounds were evaluated and compared with control probiotic cells. Five micro liters of diluted cDNA (1:20) and 100 nM of respective primer were used in each real-time RT-PCR using SYBR Green Supermix (Bio-Rad, Hercules, CA, USA) with an Mx3000P instrument (Stratagene, Cedar Creek, TX, USA). The RT-PCR conditions were optimized based on the analysis of melting points of the primers. The relative changes in gene expression in with treated cells compared with the untreated control were calculated using the 2^{-ΔΔ}CT method.

### EXAMPLE 6

The response of probiotic cells from interactions with gastric pH and bile salts was also characterized to demonstrate the protective effects of infused polyphenols. The ability to maintain intracellular pH homeostasis is vital for cell viability. The roles of infused phenolic bioactives in influencing changes in the intracellular pH upon exposure to gastric conditions were measured using ratiometric fluorescence imaging of bacteria stained with a pH sensitive fluorescent probe 5 (and 6)-carboxyfluorescein diacetate N-succimidyl ester (cFDASE). For this analysis, the bacteria were stained by incubating for 15 min at 30° C. in the presence of 10.0 μM cFDASE before treatments and the stained cells were infused with phenolic bioactives. Cells with and without infused phenolic bioactives were treated with the SGF conditions. Intracellular pH (pH_{In}) of probiotic cells after the simulated digestion was measured.

To assess changes in the fluorescence properties of pH sensitive dyes, fluorescence intensities of cells were measured at excitation wavelengths of 490 and 440 nm by using two laser sources in a confocal microscope. The emission signal was measured using a band-pass filter from 515-540 nm. The ratio fluorescence intensity of individual cells based on excitation sources of 490-and-440 nm ratios was measured before and after treatment. The results demonstrate the influence of phenolic bioactives in influencing the pH response of bacterial cells.

The interaction of bile salts with infused bacterial cells was also evaluated. Based on their surface activity, bile salts can influence the membrane permeability of bacterial cells. To evaluate interactions of bile salts with the probiotic cells with and without infused phenolic bioactives during simulated intestinal digestion, fluorescent analog of bile salts, cholyl-L-lysyl-fluorescein (CLF) were used during simulated intestinal digestion. CLF is a well-known fluorescent bile salt analog that has been used in prior studies to illustrate binding of bile salts to cells and tissues. The interaction of probiotic cells and fluorescent derivative of bile salt was analyzed using multiphoton fluorescence microscopy. Images were collected using a 488 nm excitation and 525/25 bandpass emission filter. In addition, changes in the cell membrane permeability of the phenolic infused probiotics and control cells were measured using a nucleic acid binding, membrane impermeable PI dye.

Stability of probiotics against gastric pH and bile salt tolerance is shown to be enhanced by the infusion of a diverse class phenolic compounds (catechins, fisetin, curcumin) and phenolic rich plant extracts (grape seed extract, fruit extract from jaboticaba etc.) in cells. The protection is not achieved by simple mixture of the phenolic compounds with cells. Probiotic cells infused with phenolic bioactives will survive passage through the GI tract and based on improved muco-adhesion properties, these modified probiotic cells will have higher persistence in the gut compared to the controls. Furthermore, the combination of probiotic activity of phenolic bioactive infused bacteria and generation of phenolic metabolites will significantly increase the availability of beneficial polyphenols in the gut.

### EXAMPLE 7

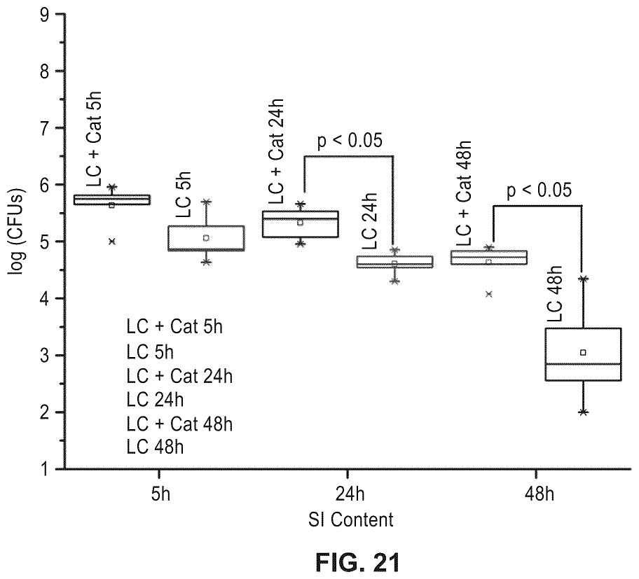

The persistence of phenolic infused probiotic bacteria in-vivo was evaluated using a mouse model. Survivability, persistence and mucoadhesion of cells with and without infused catechin were evaluated by plating serially diluted contents and tissue collected from different sections of gastrointestinal tract of each mouse as shown in FIG. 21 through FIG. 25.

Six-week-old C57BL/6J male mice were purchased from Jackson Laboratories, Sacramento, CA. Mice were fed standard chow diet for a week followed by a semi-synthetic diet for 4 consecutive days before the experiment. Mice (n=5 for each control and sample) were fasted for 12 h before oral gavage a single dose of model probiotic L. casei strain (8 log CFUs) with and without infused catechin. For in-vivo experiments, L. casei strain were serially cultured in rifampicin supplemented MRS broth to develop rifampicin resistant strain and enumerated using rifampicin added MRS agar plates. After oral gavage, mice were sacrificed at 5 h, 24 h and 48 h time intervals to extract the contents from small intestine, cecum and colon. Ilium, cecum and colon tissues were also collected after extracting the contents. The contents (100 mg) from each gastrointestinal sections were immediately plated on MRS agar plates after serial dilution using 1×PBS and plates were incubated at 37° C. for 48 h. Tissue sections were soaked in sterilized 1×PBS and grinded using sterilized tissue grinder. The mixtures were immediately plated on MRS agar as mentioned above.

FIG. 21 through FIG. 25 plot cell counts for catechin infused bacterial cells compared to the controls observed in the small intestine (SI), cecum, colon, contents and ilium tissue and cecum tissue at time points 5, 24 and 48 hours.

The results of the in-vivo study demonstrate significant (90% (1 log) or more) enhancement in in-vivo persistence of catechin infused Lactobacillus casei strain compared to the control bacteria without infused phenolic compound. FIG. 21 to FIG. 23 and FIG. 24 and FIG. 25 also show that after 5 hours of oral gavage of cells (9 log CFUs/mouse) with and without infused catechin, the distribution of cells was significantly high (2-3 log CFUs) in cecum and colon in comparison to small intestine.

Overall, the results show significantly (90% or more) higher levels of cell count for catechin infused bacterial cells compared to the controls and these differences were observed in the small intestine, cecum and colon sections of the GI tract at all time points (5, 24 and 48 hours). Moreover, the cells infused with catechin had significantly higher attachment to the tissues of ilium and cecum sections of the gut compared to the control bacteria. This result suggests enhanced mucoadhesion of bacterial cells with infused catechin.

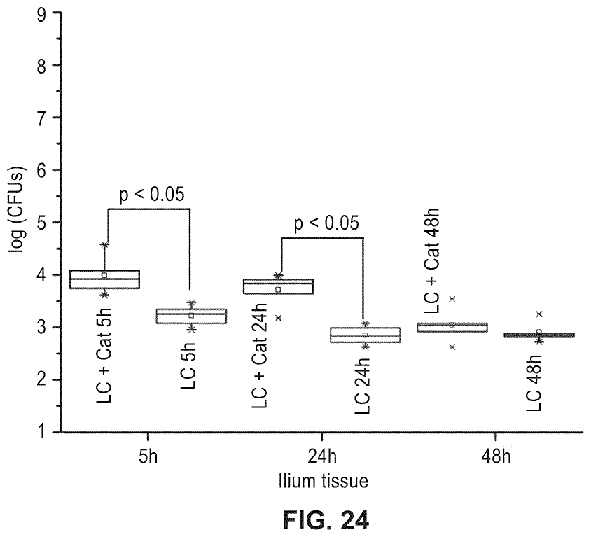

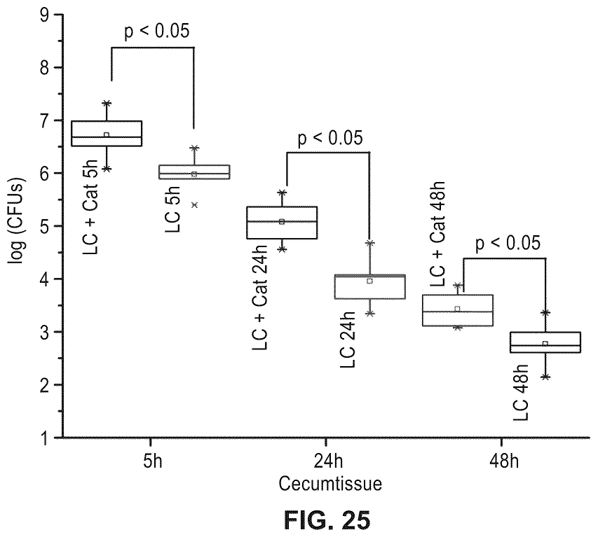

Survivability of cells with infused catechin were not significantly different than that of cells without infused catechin. However, retention of cells with infused catechin after 24 h and 48 h in each section was significantly higher in comparison to the retention of cells without infused cells. Moreover, the cells with infused catechin enumerated after grinding tissue of ilium and cecum of the mouse are significantly different than log CFUs of the cells without infused catechin as seen in FIG. 24 and FIG. 25. This corroborates the significantly enhanced mucoadhesion of cells with infused catechin. Enhanced persistence and mucoadhesion of cells with infused model plant extract might be due to increased cell surface hydrophobicity with infused plant extract or due to surface plant extract interactions with mucin biopolymer.

Based on the in-vivo results, further animal studies were conducted with two selected compositions of phenolic bioactive infused probiotics (combination of phenolic bioactives and probiotics). The controls were the selected probiotics without infusion of phenolic compounds. For in-vivo delivery, mice were divided into 3 sub-groups of 5 (alpha=0.05, beta=0.2 and power=0.9) mice for each time point and for each selected composition. The total number of mice was 100 with two compositions and 4 time points (5, 12, 24 and 48 h). This number of mice was selected based on power analysis of the preliminary results.

For this in-vivo illustration, C57BU6J male mice were individually housed in shoebox cages at 20° C. to 22° C. and 50±10% humidity levels. The gender of mice was maintained constant to avoid gender related differences. The mice had ad libitum access to feed and weighed once a week. Mice were fasted for 8-10 h before being fed the samples. Single dose of probiotic bacteria with and without infused phenolic extract were orally gavaged to mice. Mice were given a regular diet 1 hour after oral administration of the samples. Small intestine, cecum and colon content were collected after 5, 24, 48, and 72 h of probiotic administration from each of the treatment and control groups to evaluate persistence the gut. Tissue from these sections was also collected to evaluate muco-adhesion and colonization of probiotic bacteria.

The cecum and colon contents (50 mg) will also be collected after administration of probiotics for each time point of each individual group will be immediately soaked in 1×PBS for 1 h at 4° C. for analysis of in-vivo persistence and colonization of delivered probiotic microbes.

The content flushed out from the small intestine was pelleted by centrifuging at 5000 rpm for 5 min before dispersing 50 mg of each samples in 1×PBS. The samples soaked in 1×PBS were vortexed to disperse the content. The dispersed samples were serially diluted using 1×PBS, plated on the respective agar plates and incubated at 37° C. in GasPak Anaerobe Pouches for 48-72 h before enumeration.

For muco-adhesion and colonization, probiotic cells were extracted from the tissue sections and adhered mucus layer. For isolation of bacterial cells, the tissue sections were ground using a closed chamber tissue grinder system in the presence of 1×PBS (1 ml/gm of tissue). Mixtures were serially diluted and enumerated by spreading on the respective plates in similar manner as done with the contents. The results compared the delivery of probiotic bacteria with and without infused plant extracts.

### EXAMPLE 8

Simulated colonic fermentation was used to characterize the metabolites generated by phenolic infused probiotics. The controls in this experiment will include cells without phenolics and cells incubated with phenolic bioactives in the extracellular medium. In-vitro simulated colonic fermentation experiments were conducted in a simulated colonic environment for 24 hours under anaerobic conditions. The simulated ileal efflux media (SIEM) without added polysaccharides was used. This media included bactopeptone, salts, casein, cysteine, plus vitamins.

Growth of the selected bacterial strains was measured using the standard plate counting methods. Based on the colonic fermentation, the growth of probiotic strains with infused phenolic bioactives, control cells without phenolic bioactives and cells with phenolic bioactives in the extracellular media were quantified using the standard plating methods. Concentration of short chain fatty acids (SCFAs; acetate, butyrate and propionate), branched-chain fatty acids (BCFAs; isobutyrate, valerate and isovalerate) and intermediate metabolites (lactate and formate) in the fermented extracts were analyzed using gas chromatography analysis. Concentrations were calculated on the basis of a standard calibration curve for each of the different SCFA or BCFA. Untargeted phenolic metabolites analyses were carried out using TripleTOF® 6600 Quadrupole Time-Of-Flight mass spectrometer (Sciex) coupled with a high-performance liquid chromatography (HPLC). A 200 μm×0.5 mm trap column and a 75 μm×15 cm analytical column (ChromXP C18-CL, 3 μm, 120 Å; Sciex) were used.

The mobile phases were at 0.1% formic acid in water (A) and 0.1% formic acid in acetonitrile (B). A flow rate was 300 nL/min. A LC gradient elution condition was initially 5% B to 30% B (90 min) and 80% B (95-105 min). The electrospray ionization was performed in both positive and negative ion modes. The mass spectra was acquired from 1000-8000 m/z with an accumulation time of 0.1 s in high sensitivity mode. The injection volume was 5 μL and the resuspension volume was set to 100 μL. The source parameters were optimized to obtain reproducible mass data.

The complete data processing was performed with MS-Dial 2.70 software. The metabolites were evaluated comparing the peaks of known compounds or a library of compounds. In addition, changes in microbial metabolites including sugars, amino acids and microbial-derived byproducts were assessed using NMR spectroscopy methods.

### EXAMPLE 9

In order to further understand the impact of the encapsulant on the cellular metabolism, metabolomics analyses were conducted using a high-performance liquid chromatography coupled with tandem mass spectrometry (HPLC-MS/MS) to identify potential curcumin metabolites produced by the cell carrier, and to characterize the intracellular and extracellular metabolic profiles of alive cell carriers after encapsulation. Defined as a comprehensive analysis of all metabolites present in a biological system, metabolomic analyses have been widely applied to investigate changes in the metabolic activities in response to specific stimuli or genetic modifications. Among all the analytical platforms, HPLC-MS/MS remains as a popular and powerful technique due to its high throughput, soft ionization, and good coverage of metabolites.

Curcumin metabolites were identified using a mass-based search followed by manual verification. The semi-quantitative comparison was achieved based on peak heights. The control cells and cells incubated with extracellular curcumin were analyzed with the same protocol.

The MS/MS analysis identified remaining curcumin and key curcumin metabolites produced by cell carriers. Cells encapsulated with curcumin showed more curcumin content intracellularly than cells incubated extracellularly with the same starting curcumin concentration (52.7 ug/ml). This might be attributed to the cellular structure and components that stabilized the compound within the cell. Similarly, extracellular curcumin was only observed in the sample cells incubated with curcumin in the medium.

Curcumin content in the medium was below limit of detection for cells encapsulated with the compound, which indicated that curcumin was stabilized in the cell carrier without leaking to the medium during the extended period of incubation. The robust encapsulation system is favorable to achieve controlled release and provide protection through the complex gastrointestinal tract.

The identified metabolites produced from curcumin included tetrahydrocurcumin and bisdemethoxycurcumin. The pathways of synthesizing these metabolites have been identified and the reaction is mostly mediated by NADPH-dependent curcumin/dihydrocurcumin reductase. Interestingly, different levels of metabolites were produced with different incubation condition. Tetrahydrocurcumin was produced from both encapsulated curcumin and extracellular curcumin and localized mostly intracellularly. Bisdemethoxycurcumin was produced by encapsulated curcumin intracellularly and was not detected outside of the cell carrier in both conditions.

Characterization of the synthesis pathways could be undertaken, but these observations indicate that it is possible to select different incubation conditions to promote the production of target metabolites depending on the interaction between the live cell carrier and the target compounds and the specific production mechanism.

In addition to curcumin-derived metabolites, overall metabolomic profiles of the cell carriers under different incubation conditions with curcumin were also evaluated to assess the impact of encapsulation on the live cell carrier. The metabolites with statistically significant variation in content between the control cells (Tmt1: control), cells encapsulated curcumin (Tmt2: encapsulated curcumin), cells incubated with extracellular curcumin (Tmt3: extracellular curcumin) (p-value δ 0.01) were evaluated. The quantification was normalized by subtracting the mean value of each metabolites across treatments and dividing by standard deviation to focus on the different effect caused by the experiments.

For extracellular metabolites, most compounds were at a lower concentration compared to the control group. Tmt3 showed exceptions that a few metabolites were at the highest concentration level. As for the intracellular metabolites, the same pattern is observed, in which most compounds showed significantly lower concentration compared to the control. This might indicate that while the cell still possessed metabolism capability, the overall metabolism was depressed by the phenolic compound (located either intracellularly or extracellularly) and/or the encapsulation process for Tmt2. A few metabolites spiked depending on the treatment group, which might account for the metabolites from curcumin, and/or the stress and defense mechanism of the cell.

Partial least square discriminant analysis (PLS-DA) is one of the well-established classification techniques in metabolomics studies and was used to evaluate the overall changes of the cell carrier's metabolic profile. A comprehensive metabolic profile of more than 3300 identified intracellular and extracellular metabolites identified for Tmt1, Tmt2 and Tmt3 were respectively analyzed using PLS-DA, and the relation and separation between the difference groups was observed. This process indicated that the overall, intracellular metabolite profile of the two treatment groups that were exposed to curcumin were statistically changed compared to the control group (p-value δ 0.05).

As for the extracellular metabolites, cells with encapsulated curcumin presented a different metabolite profile compared to the control cell. These observations demonstrated that both intracellularly and extracellularly, the metabolism of cell carriers that are exposed to curcumin (either from outside or within the cell) is modified and moved to the same direction, which are consistent with the information obtained previously. Moreover, despite the overall potentially metabolic depression effect caused by the encapsulation process and the encapsulated bioactive compounds, the cell carriers were able to produce metabolites both intracellularly and extracellularly.

Accordingly, the simultaneous delivery of phenolic compounds (PC) and probiotics may improve the proliferation of probiotics and the absorption of PC and their metabolites in the gut. However, probiotic cells may be sensitive to acid condition and bile salts, hampering the survivability. This study focused on the vacuum infusion of polyphenols from plant extracts in live probiotic cells as an alternative to improve the viability of probiotics during in vitro digestion test.

In addition, the metabolic activity of cells was evaluated by fluorescence using resazurin, as well as the hydrophobicity of probiotic cells. Vacuum infusion of polyphenols increased the hydrophobicity of probiotic cells, which is expected to improve their binding in the gut. After the addition of simulated gastric fluid, control cells, without infusion of polyphenols, showed a reduction around 3 log CFU. On the other hand, probiotics infused with plant extracts or catechin maintained their population. Furthermore, probiotic cells after infusion were metabolically active. This approach eliminated the need of probiotic encapsulation to get high amounts of cells in the gut.

From the description herein, it will be appreciated that the present disclosure encompasses multiple implementations of the technology which include, but are not limited to, the following:

A method for enhancing probiotic viability from exposure to gastric acid and intestinal bile salt environments, the method comprising: (a) providing one or more strains of living beneficial probiotic bacteria cells; (b) providing a purified polyphenolic compound or a mixture of polyphenolic compounds; and (c) infusing said polyphenolic compounds into the cell membranes and/or interiors of said probiotic bacteria cells; (d) wherein hydrophobicity of cell membrane surfaces increases cell adhesion to gut walls; and (e) wherein persistence of said infused probiotic bacteria cells in a gut of a subject is enhanced.

The method of any preceding or following implementation, wherein said probiotic bacteria cells are selected from the group consisting of Lactobacillus, Bacteriodes and Bifidobacteria bacterial cells.

The method of any preceding or following implementation, wherein said probiotic bacteria cells are one or more of the group of Lactobacillus bacterial cells consisting of L. paracasei, L. casei, L. rhamnosus. L. acidophilus and L. plantarum.

The method of any preceding or following implementation, wherein said polyphenolic compounds are hydrophilic or hydrophobic plant extracts.

The method of any preceding or following implementation, wherein said polyphenolic compounds are compounds selected from the group of curcumin, procyanidins, quercetin, isoflavones and catechins.

The method of any preceding or following implementation, wherein said polyphenolic compounds are compounds selected from the group of flavonoids and their glycol conjugates (anthocyanin, proanthocyandins, isoflavones), phenolic acids (ferulic acid, gallic acids and others), curcumoids and derivatives, and stilbenes (resveratrol).

The method of any preceding or following implementation, wherein said polyphenolic compounds are compounds extracted from the group of grape, jaboticaba and guarana seeds.

The method of any preceding or following implementation, wherein said polyphenolic compounds are compounds extracted from the group of berries, pomegranates, other phenolic rich fruits and their skin and seed extracts.

The method of any preceding or following implementation, wherein said infusion takes place in an approximately 1% to approximately 35% ethanol solution.

The method of any preceding or following implementation, wherein said infusion takes place in a vacuum or a combination of vacuum and positive external pressure environment.

The method of any preceding or following implementation, wherein the vacuum pressure (e.g., negative pressure) is at least about 3 Torr.

The method of any preceding or following implementation, wherein a mixture of probiotic bacterial cells and polyphenolic compounds are exposed to 99% vacuum pressure (e.g., negative pressure) for about 4 seconds to about 7 seconds.

The method of any preceding or following implementation, further comprising: metabolizing said infused polyphenolic compounds to produce biologically active metabolites.

A method for enhancing probiotic viability from exposure to gastric acid and intestinal bile salt environments, the method comprising: (a) providing one or more strains of living beneficial probiotic bacteria cells; (b) extracting polyphenolic compounds from a source to produce an extract solution of polyphenolic compounds; (c) mixing the probiotic bacteria cells with the extract solution; and (d) applying vacuum pressure to the mixed solution to infuse said polyphenolic compounds into cell membranes and/or interiors of said probiotic bacteria cells.

The method of any preceding or following implementation, wherein said probiotic bacteria cells are selected from the group consisting of Lactobacillus, Bacteriodes and Bifidobacteria bacterial cells.

The method of any preceding or following implementation, wherein said probiotic bacteria cells are one or more of the group of Lactobacillus bacterial cells consisting of L. paracasei, L. casei, L. rhamnosus. L. acidophilus and L. plantarum.

The method of any preceding or following implementation, wherein said polyphenolic compounds are extracted with an approximately 1% to approximately 35% ethanol solution.

The method of any preceding or following implementation, wherein said polyphenolic compounds are hydrophilic or hydrophobic plant extracts.

The method of any preceding or following implementation, wherein said polyphenolic compounds are compounds extracted from one or more of the group of grape, jaboticaba and guarana seeds.

The method of any preceding or following implementation, wherein a mixture of probiotic bacterial cells and polyphenolic compounds are exposed to 99% vacuum pressure (e.g., negative pressure) for about 4 seconds to about 7 seconds.

The method of any preceding or following implementation, wherein the mixture solution is subjected to multiple iterations of vacuum pressure or vacuum pressure and positive external pressure.

The method of any preceding or following implementation, further comprising: (a) collecting infused bacterial cells; (b) extracting polyphenolic compounds from a second source with an ethanol solution to produce a second extract solution of polyphenolic compounds; (c) mixing the infused probiotic bacteria cells with a second extract solution of a polyphenolic compounds; and (d) applying vacuum pressure to the mixed solution to infuse said second polyphenolic compounds into cell membranes and/or interiors of said probiotic bacteria cells.

An engineered probiotic bacteria, comprising: (a) one or more strains of living beneficial probiotic bacteria cells, said bacteria cells with cell membranes and/or interiors infused with exogenous polyphenolic compounds; (b) wherein hydrophobicity of said cell membrane surfaces increases cell adhesion to gut walls; and (c) wherein persistence of said infused probiotic bacteria cells in a gut of a subject is enhanced.

The engineered probiotic bacteria of any preceding implementation, further comprising: a second polyphenolic compound from a second source infused in said cell membranes and/or cell interiors.

As used herein, term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these group elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system or method.

As used herein, the term “set” refers to a collection of one or more objects. Thus, for example, a set of objects can include a single object or multiple objects.

Relational terms such as first and second, top and bottom, and the like may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential features or elements of the technology describes herein or any or all the claims.

In addition, in the foregoing disclosure various features may grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after that application is filed. Accordingly the reader should consult the application as filed for the original content of the disclosure. Any deletion of content of the disclosure should not be construed as a disclaimer, forfeiture or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

TABLE 1
Total protein content and hydrophobicity of probiotic cells before and after vacuum infusion.

| | | | | | Total |
|---|---|---|---|---|---|
| | | Hydrophobicity | Hydrophobicity | Hydrophobicity | protein |
| | Hydrophobicity | after VI of | after VI of | after VI of | content |
| Cells | (%) | 25% ethanol | catechin | GSE | (mg/g) |
| L. paracasei | 6.8 ± 2.0 | 6.8 ± 1.7 | 24.2 ± 3.5 | 40.3 ± 2.6 | 158 ± 13 |
| L. casei | 5.7 ± 1.7 | 2.2 ± 0.2 | 4.4 ± 0.6 | 24.5 ± 1.5 | 128 ± 9 |
| L. rhamnosus | 16.0 ± 3.1 | 9.9 ± 0.5 | 22.1 ± 4.4 | 33.7 ± 2.3 | 177 ± 9 |

What is claimed is:

1. A method for enhancing probiotic viability from exposure to gastric acid and intestinal bile salt environments, the method comprising:
(a) providing one or more strains of living beneficial probiotic bacteria cells;
(b) providing a purified polyphenolic compound or a mixture of polyphenolic compounds; and
(c) infusing said polyphenolic compounds into the cell membranes and/or interiors of said probiotic bacteria cells;
(d) wherein hydrophobicity of cell membrane surfaces increases cell adhesion to gut walls; and
(e) wherein persistence of said infused probiotic bacteria cells in a gut of a subject is enhanced.

2. The method of claim 1, wherein said probiotic bacteria cells are selected from the group consisting of Lactobacillus, Bacteriodes and Bifidobacteria bacterial cells.

3. The method of claim 2, wherein said probiotic bacteria cells are one or more of the group of Lactobacillus bacterial cells consisting of L. paracasei, L. casei, L. rhamnosus. L. acidophilus and L. plantarum.

4. The method of claim 1, wherein said polyphenolic compounds are hydrophilic or hydrophobic plant extracts.

5. The method of claim 1, wherein said polyphenolic compounds are compounds selected from the group of curcumin, procyanidins, quercetin, isoflavones and catechins.

6. The method of claim 1, wherein said polyphenolic compounds are compounds selected from the group of flavonoids and their glycol conjugates (anthocyanin, proanthocyandins, isoflavones), phenolic acids (ferulic acid, gallic acids and others), curcumoids and derivatives, and stilbenes (resveratrol).

7. The method of claim 1, wherein said polyphenolic compounds are compounds extracted from the group of grape, jaboticaba and guarana seeds.

8. The method of claim 1, wherein said polyphenolic compounds are compounds extracted from the group of berries, pomegranates, other phenolic rich fruits and their skin and seed extracts.

9. The method of claim 1, wherein said infusion takes place in an approximately 1% to approximately 35% ethanol solution.

10. The method of claim 1, wherein said infusion takes place in a vacuum or a combination of vacuum and positive external pressure environment.

11. The method of claim 10, wherein the vacuum pressure (e.g., negative pressure) is at least about 3 Torr.

12. The method of claim 10, wherein a mixture of probiotic bacterial cells and polyphenolic compounds are exposed to 99% vacuum pressure (e.g., negative pressure) for about 4 seconds to about 7 seconds.

13. The method of claim 1, further comprising:
metabolizing said infused polyphenolic compounds to produce biologically active metabolites.

14. A method for enhancing probiotic viability from exposure to gastric acid and intestinal bile salt environments, the method comprising:
(a) providing one or more strains of living beneficial probiotic bacteria cells;
(b) extracting polyphenolic compounds from a source to produce an extract solution of polyphenolic compounds;
(c) mixing the probiotic bacteria cells with the extract solution; and
(d) applying vacuum pressure to the mixed solution to infuse said polyphenolic compounds into cell membranes and/or interiors of said probiotic bacteria cells.

15. The method of claim 14, wherein said probiotic bacteria cells are selected from the group consisting of Lactobacillus, Bacteriodes and Bifidobacteria bacterial cells.

16. The method of claim 15, wherein said probiotic bacteria cells are one or more of the group of Lactobacillus bacterial cells consisting of L. paracasei, L. casei, L. rhamnosus. L. acidophilus and L. plantarum.

17. The method of claim 14, wherein said polyphenolic compounds are extracted with an approximately 1% to approximately 35% ethanol solution.

18. The method of claim 14, wherein said polyphenolic compounds are hydrophilic or hydrophobic plant extracts.

19. The method of claim 14, wherein said polyphenolic compounds are compounds extracted from one or more of the group of grape, jaboticaba and guarana seeds.

20. The method of claim 14, wherein a mixture of probiotic bacterial cells and polyphenolic compounds are exposed to 99% vacuum pressure (e.g., negative pressure) for about 4 seconds to about 7 seconds.

21. The method of claim 14, wherein the mixture solution is subjected to multiple iterations of vacuum pressure or vacuum pressure and positive external pressure.

22. The method of claim 14, further comprising:
(a) collecting infused bacterial cells;
(b) extracting polyphenolic compounds from a second source with an ethanol solution to produce a second extract solution of polyphenolic compounds;
(c) mixing the infused probiotic bacteria cells with a second extract solution of a polyphenolic compounds; and
(d) applying vacuum pressure to the mixed solution to infuse said second polyphenolic compounds into cell membranes and/or interiors of said probiotic bacteria cells.

23. An engineered probiotic bacteria, comprising:
(a) one or more strains of living beneficial probiotic bacteria cells, said bacteria cells with cell membranes and/or interiors infused with exogenous polyphenolic compounds;
(b) wherein hydrophobicity of said cell membrane surfaces increases cell adhesion to gut walls; and
(c) wherein persistence of said infused probiotic bacteria cells in a gut of a subject is enhanced.

24. The engineered bacteria of claim 22, further comprising:
a second polyphenolic compound from a second source infused in said cell membranes and/or cell interiors.
